(19) United States
(12) Patent Application Publication
(10) Pub. No.: US 2021/0244936 A1
(43) Pub. Date: Aug. 12, 2021

(54) FLEXIBLE MULTILAYERED PUMP FOR DRIVING BIOLOGICAL FLUID

(71) Applicant: COLORADO STATE UNIVERSITY RESEARCH FOUNDATION, Fort Collins, CO (US)

(72) Inventor: David L. BARK, Fort Collins, CO (US)

(21) Appl. No.: 16/973,081

(22) PCT Filed: Jun. 5, 2019
(86) PCT No.: PCT/US19/35480

§ 371 Date: Dec. 8, 2020

Related U.S. Application Data
(60) Provisional application No. 62/682,389, filed on Jun. 8, 2018.

(51) Int. Cl. A61M 60/515; A61N 1/04; A61M 60/152; A61M 60/178; A61M 60/268; A61M 60/896; A61M 60/837; A61M 60/427

(52) U.S. Cl. CPC A61M 60/515; A61N 1/0404; A61M 60/152; A61M 60/178; A61M 60/268; A61M 2205/52; A61M 60/837; A61M 60/427; A61M 2205/04; A61M 2205/3327; A61M 60/896

(57) ABSTRACT

An example pump for driving a biological fluid is described herein. The pump can include an inner tubular structure and an outer tubular structure arranged around the inner tubular structure. The outer tubular structure can be configured as an artificial muscle. The pump can also include a gel layer disposed between the inner and outer tubular structures.

### CROSS-REFERENCE TO RELATED APPLICATIONS

This application claims the benefit of U.S. provisional patent application No. 62/682,389, filed on Jun. 8, 2018, and entitled “FLEXIBLE MULTILAYERED PUMP FOR DRIVING BIOLOGICAL FLUID,” the disclosure of which is expressly incorporated herein by reference in its entirety.

### BACKGROUND

Cardiovascular disease has remained the leading cause of death for over a century in the United States. One result has been an increasing number of ventricular assist devices (VADs) being utilized to treat patients with various forms of heart failure, affecting 23 million people worldwide. A heart transplant is the primary solution for many of these patients. However, fewer than 3820 heart transplants are performed in the world each year due to an insufficient number of available viable hearts, despite a much larger need. Due to the need for alternative, VADs were developed as mechanical pumps that enhance or replace the function of the ventricle. Continuous-flow VADs are the mostly commonly used and consist of a driveline connected through the skin of a patient to control a mechanical impeller rotating inside a housing to pump blood. Despite substantial improvements in VADs (especially durability), a larger application of the technology has been limited due to clinically significant adverse events that include bleeding and thrombosis issues.

One of the most common early adverse events with patients receiving VADS is nonsurgical bleeding, especially in the gastrointestinal tract. High non-physiological shear stress and reduced pulse pressure may be involved. Shear stress in a continuous flow VAD is complex due to the fast rotations of the impeller, with regions that can exceed 500 dyne/cm². Comparatively, the highest physiological shear stress is ˜120 dynes/cm². High shear stress can lead to acquired von Willebrand Syndrome (aVWS)—a loss of high molecular weight von Willebrand Factor (VWF) multimers, which occurred in all patients receiving left VADs in at least one study. High shear stress can also lead to platelet receptor shedding and a reduction in platelet aggregation for patients with a continuous flow VAD. As opposed to directly effecting the cells and proteins involved in hemostasis, lack of pulse pressure in these VADs, specifically, can lead to hypoperfusion and angiodysplasia, while further influencing arterial remodeling in response to altered mechanical stress. Overall, the mechanism for bleeding can be multifactorial and remains unclear, yet what is clear is that non-physiological shear stress in VADs, potentially combined with continuous flow leads to bleeding complications.

In addition to bleeding, thromboembolic complications are another major concern for VADs. Thrombus formation, an undesired hemostatic response, can occur in regions involving nonphysiological flow. High shear stress and shear gradients are reported to promote rapid thrombus growth, along with hemolysis and VWF self-association. Hemoloysis can lead to platelet activation, while VWF, can enhance platelet accrual since it is a key protein for platelet capture at high shear stress. Although recent advances in continuous flow pumps have increased the probability of survival, the number of patients experiencing device thrombosis or ischemic stroke remain high. This demonstrates that non-physiologically high shear stress associated with VADs can lead to thrombotic responses.

### SUMMARY

An example pump for driving a biological fluid is described herein. The pump can include an inner tubular structure and an outer tubular structure arranged around the inner tubular structure. The outer tubular structure can be configured as an artificial muscle. The pump can also include a gel layer disposed between the inner and outer tubular structures.

Additionally, the inner tubular structure, the outer tubular structure, and/or the gel layer can be flexible. In some implementations, each of the inner tubular structure, the outer tubular structure, and the gel layer can be flexible.

Alternatively or additionally, each of the inner tubular structure, the outer tubular structure, and the gel layer can be a biocompatible material.

Alternatively or additionally, the inner tubular structure can be an extensible flexible polymer or an inextensible flexible polymer.

Alternatively or additionally, the outer tubular structure can be an inextensible flexible polymer.

Alternatively or additionally, the pump can be configured such that a maximum internal shear stress experienced by the biological fluid is about 150 dynes/cm² or less. Alternatively or additionally, the pump can be configured such that a maximum internal shear stress experienced by the biological fluid is about 60 dynes/cm² or less.

Alternatively or additionally, the gel layer can be a liquid, a liquid hydrogel, an organogel, a gelatin, an animal produced gel, or combinations thereof.

Alternatively or additionally, the gel layer can have a thickness between about 0.1-2 cm. Optionally, in some implementations, the gel layer can have a thickness between about 0.1-0.6 cm.

Alternatively or additionally, the outer tubular structure can include a plurality of pouches. Each of the pouches can be filled with fluid. Optionally, in some implementations, the pouches are spaced apart from one another in an axial direction and/or a circumferential direction along the outer tubular structure. Optionally, the pouches are arranged in a spiral pattern along the outer tubular structure. In some implementations, the outer tubular structure can include a plurality of polymer layers, and the pouches can be formed between the polymer layers. For example, the polymer layers can be joined by means including, but not limited to, thermal welding, gluing, bonding, or otherwise fastening the polymer layers together.

Alternatively or additionally, each of the pouches can be configured to deform in response to an external stimulus. For example, a deformed pouch can be configured to displace the gel layer, and the gel layer can be configured to compress the inner tubular structure when displaced by the deformed pouch.

Alternatively or additionally, the pump can include a plurality of electrode sets. Each respective electrode set can be disposed on a respective one of the pouches. For example, each respective electrode set and pouch can form an electrohydraulic transducer.

Alternatively or additionally, the pump can include an ionic polymer-metal composite (IPMC) or piezoelectric material arranged in electrical contact with each of the electrode sets.

Alternatively or additionally, the pump can include a controller operably coupled to the electrode sets. The controller can include a processor and memory operably coupled to the processor, where the memory has computer-executable instructions stored thereon that, when executed by the processor, cause the controller to stimulate one or more of the electrode sets in a predetermined pattern. In some implementations, the one or more of the electrode sets can be stimulated in the predetermined pattern to cause wavelike action in the outer tubular structure. In other implementations, the one or more of the electrode sets can be stimulated in the predetermined pattern to cause peristaltic action in the outer tubular structure. In yet other implementations, the one or more of the electrode sets can be stimulated in the predetermined pattern to cause non-peristaltic action in the outer tubular structure. In yet further implementations, the one or more of the electrode sets can be stimulated in the predetermined pattern to simultaneously contract the entire outer tubular structure.

Alternatively or additionally, the pump can include a controller operably coupled to the electrode sets. The controller can include a processor and memory operably coupled to the processor, where the memory has computer-executable instructions stored thereon that, when executed by the processor, cause the controller to stimulate one or more of the electrode sets. Additionally, stimulating the one or more of the electrode sets can cause one or more of the pouches to deform. Deforming the one or more of the pouches can cause circumferential and/or longitudinal contraction of the outer tubular structure, which can pump the biological fluid through the inner tubular structure.

Alternatively or additionally, the controller can be configured to sense mechanical strain using one or more of the electrode sets.

Alternatively or additionally, the pump can include a battery operably coupled to the electrode sets.

Alternatively or additionally, the pump can further include a pneumatic or hydraulic pump arranged in fluid connection with the pouches. Additionally, the pump can further include a controller operably coupled to the pneumatic or hydraulic pump. The controller can include a processor and memory operably coupled to the processor, where the memory has computer-executable instructions stored thereon that, when executed by the processor, cause the controller to actuate the pneumatic or hydraulic pump, where actuating the pneumatic or hydraulic pump causes one or more of the pouches to deform.

Alternatively or additionally, the pump can include at least one prosthetic valve arranged in fluid connection with the inner tubular structure. Optionally, the at least one prosthetic valve can be configured to prevent backflow of the biological fluid. In some implementations, the at least one prosthetic valve can include at least one leaflet. Optionally, the at least one leaflet can define a sinus region. Alternatively or additionally, the at least one prosthetic valve can be a trileaflet valve.

Alternatively or additionally, at least a portion of the inner tubular structure can extend outside of the outer tubular structure. Additionally, the at least one prosthetic valve can be arranged in the portion of the inner tubular structure that extends outside of the outer tubular structure.

Alternatively or additionally, the pump can be a ventricular assist device, a dialysis pump, or a blood pump.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a diagram illustrating an example pump for driving a biological fluid according to implementations described herein.

FIG. 2 is a diagram illustrating an example prosthetic trileaflet valve in the closed position according to an implementation described herein.

FIG. 3 is a diagram illustrating an example prosthetic trileaflet valve in the open position according to an implementation described herein.

FIG. 4 is a diagram illustrating example prosthetic valve design as formed on a flexible sheet according to an implementation described herein.

FIG. 5 is a block diagram of an example computing device.

FIG. 6A illustrates the embryonic heart. FIG. 6B is a diagram illustrating a flexible pump design according to an implementation described herein. FIG. 6C illustrates the large change in inner lumen diameter achieved using the flexible pump design of FIG. 6B.

FIG. 7A is a contour plot illustrating simulated strain rates (shear stress) throughout the inner tubular structure of an example flexible pump design.

FIG. 7B is a plot of velocity vectors illustrating simulated flow throughout the inner tubular structure of another example flexible pump design with a sinus region.

FIG. 8 is a diagram illustrating an example pump with pouches arranged in a spiral pattern according to an implementation described herein.

FIG. 9A is a diagram illustrating an example prosthetic valve according to an implementation described herein.

FIG. 9B is a diagram illustrating a cross-sectional view of an example pump with pouches arranged in a spiral pattern according to an implementation described herein.

FIG. 9C is a diagram illustrating pouch actuation in the pump shown in FIG. 9B.

FIG. 10A is a fluid-structure interaction (FSI) model of fluid shear stress for fluid flowing through the inner tubular structure of an example flexible pump with circumferentially aligned pouches.

FIG. 10B is an FSI model of fluid shear stress for fluid flowing through the inner tubular structure of an example flexible pump with axially aligned pouches.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. As used herein, the terms “about” or “approximately”, when used in reference to internal shear stress, mean within plus or minus 5 percent of the referenced internal shear stress. As used herein, the terms “about” or “approximately”, when used in reference to a linear dimension (e.g., gel layer thickness), mean within plus or minus 1 mm of the referenced linear dimension. While implementations will be described for ventricular assist device pumps, it will become evident to those skilled in the art that the implementations are not limited thereto, but are applicable for other blood pump applications (e.g., dialysis) and/or biological or industrial pump applications where low shear stress is desirable.

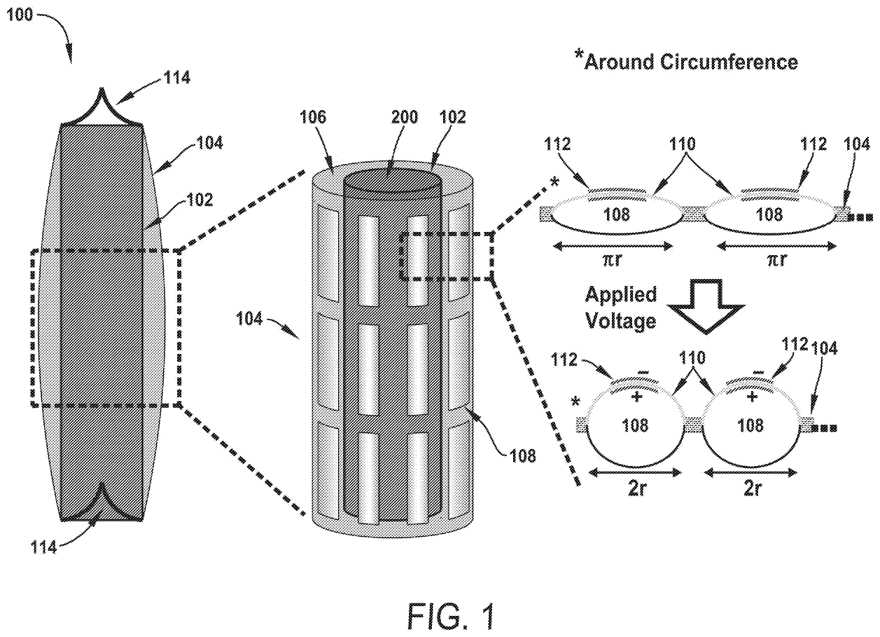

Referring now to FIG. 1, an example pump 100 for driving a biological fluid 200 is shown. As described herein, the pump 100 can be a ventricular assist device (VAD). A VAD is an electromechanical device that assists with cardiac function. A VAD can be used in a patient awaiting heart transplant and/or experiencing decreased cardiac function (e.g., heart failure). A VAD is connected between a lower heart chamber (ventricle) of the patient and the rest of the body. For example, for left VAD, the device is connected between the patient's left ventricle and the patient's aorta. For right VAD, the device is connected between the patient's right ventricle and the patient's pulmonary artery. In these implementation, the biological fluid 200 is blood. VADs are known in the art and are therefore not described in further detail below. Although VADs are described in the examples herein, this disclosure contemplates that the pump 100 can be a dialysis pump, a blood pump, or pump for driving a biological fluid other than blood. Alternatively, this disclosure contemplates that the pump 100 can be used in industrial applications. This disclosure contemplates that the pump 100 can be particularly useful in applications where low shear stress (e.g., less than about 150 dynes/cm²) is desirable.

As shown in FIG. 1, the pump 100 can include an inner tubular structure 102 and an outer tubular structure 104 arranged around the inner tubular structure 102. The outer tubular structure 104 can be configured as an artificial muscle. The pump 100 can also include a gel layer 106 disposed between the inner and outer tubular structures 102, 104. Additionally, the inner tubular structure 102, the outer tubular structure 104, and/or the gel layer 106 can be flexible. In some implementations, each of the inner tubular structure 102, the cuter tubular structure 104, and the gel layer 106 can be flexible. Optionally, each of the inner tubular structure 102, the outer tubular structure 104, and the gel layer 106 can be made of a biocompatible material. Alternatively or additionally, the inner tubular structure 102 can optionally be made of an extensible flexible polymer or an inextensible flexible polymer. Alternatively or additionally, the outer tubular structure 104 can optionally be made of an inextensible flexible polymer such as transparent biaxially-oriented polypropylene (BOPP) or linear low-density polyethylene. It should be understood that BOPP and linear low-density polyethylene are provided as examples and that this disclosure contemplates that the tubular structures can be made of other materials including, but not limited to, other polymers or silicone, or silicone rubber.

The gel layer 106 can be a liquid (e.g., saline, phosphate buffer solution, Tyrodes solution, or liquid osmotically balanced with blood), a liquid hydrogel, an organogel, a gelatin, an animal produced gel, including combinations thereof. It should be understood that these materials are only provided as examples. This disclosure contemplates that the gel layer 106 can be made of other materials. Alternatively or additionally, the gel layer 106 can have a thickness between about 0.1-2 cm (e.g., 0.10 cm, 0.11 cm, 0.12 cm . . . 1.98 cm, 1.99 cm, 2.00 cm) and any value or range therebetween. Optionally, in some implementations, the gel layer 106 can have a thickness between about 0.1-0.6 cm (e.g., 0.10 cm, 0.11 cm, 0.12 cm . . . 0.58 cm, 0.59 cm, 0.60 cm) and any value or range therebetween. The example ranges noted above are based on a relatively simplified calculation that assumes the conservation of mass for the cardiac jelly:

$$SV ≅ π {{L}_{d} ( {r}_{d} + t )}^{2} ( 1 - \frac{{L}_{s}}{{L}_{d}} {λ}^{2} ) ,$$

where SV is the stroke volume, L_{d} is the diastolic length, Lₛ is the systolic length, r_{d} is the inner tube diastolic radius, t is the gel layer thickness, and λ is the ratio of the outer radius during systole relative to diastole. Without a gel layer, λ would need to be ˜0.71 to achieve a physiologic stroke volume and end diastolic volume with a tube length of 15 cm. With a gel layer of 1 cm, λ can be ˜0.9. There are diminishing returns for increased thickness, which simultaneously leads to increased bulk in the pump, making it less suitable for implantation. These values assume a diastolic radius in the range of commercially available prosthetic heart valves. It should be understood that these ranges are only provided as examples. This disclosure contemplates that the gel layer 106 can have a thickness other than those described above, for example, a thickness between about 0.5-8 cm or between about 1.0-4.0 cm. As described herein, the presence of the gel layer 106 results in a much larger reduction in the diameter of the inner tubular structure 102 as compared to when a single tubular structure is used.

As shown in FIG. 1, the outer tubular structure 104 can include a plurality of pouches 108. Each of the pouches 108 can be filled with fluid. Optionally, the pouches 108 are spaced apart from one another in an axial direction and/or a circumferential direction along the outer tubular structure 104. As shown in FIG. 1, the pouches 108 are arranged in a grid-like pattern on the outer tubular structure 104. It should be understood that the size/shape of the pouches 108, arrangement, and/or spacing between the pouches 108 in FIG. 1 are provided only as an example. This disclosure contemplates that the size/shape of the pouches 108, arrangement, and/or spacing between the pouches 108 can be different than the example shown in FIG. 1. In some implementations, the outer tubular structure 104 can optionally include a plurality of polymer layers, and the pouches 108 can be formed between the polymer layers. The pouches 108 (e.g., size, shape, arrangement, etc.) can be designed by joining two polymer layers, For example, the polymer layers can be joined by means including, but not limited to, thermal welding, gluing, bonding, or otherwise fastening the polymer layers together. Alternatively, in some implementations, the inner tubular structure 102 and/or the outer tubular structure 104 can optionally be formed using a three-dimensional (3D) printer.

Each of the pouches 108 can be configured to deform in response to an external stimulus. For example, a deformed pouch 108 can be configured to displace the gel layer 106, and the gel layer 106 can be configured to compress the inner tubular structure 102 when displaced by the deformed pouch 108. As described herein, the presence of the gel layer 106 results in a much larger reduction in the diameter of the inner tubular structure 102 as compared to when a single tubular structure is used. For example, in the example shown in FIG. 1, the inner volume (i.e., the volume defined within the inner tubular structure 102) can reduce by a factor of t/R(1/λ²−1) (2+t/R) more than if the gel layer 106 was absent for a given λ from the pump 100, where t is the thickness of the gel layer 106, and R is radius of the inner tubular structure 102. Additionally, λ≅2/π defines the approximate circumference change based on pouch length change (assuming flattening during relaxation and full rounding during expansion).

The pump 100 can include a plurality of electrode sets 112. Each electrode set 112 can include a pair of electrodes, e.g., terminals to which a voltage or current signal can be applied and/or detected. Each respective electrode set 112 can be disposed on a respective one of the pouches 108. In some implementations, an ionic polymer-metal composite (IPMC) 110 can be arranged in electrical contact with each of the electrode sets 112. As described herein, the IPMC 110 can be activated by stimulating one or more of the electrode sets 112. It should be understood that IPMC 110 is provided only as an example. This disclosure contemplates that other electrically active materials including, but not limited to, piezoelectric materials or electromagnets can be arranged in electrical contact with each of the electrodes sets 112. Accordingly, each respective electrode set 112 and pouch 108 can form an electrohydraulic transducer.

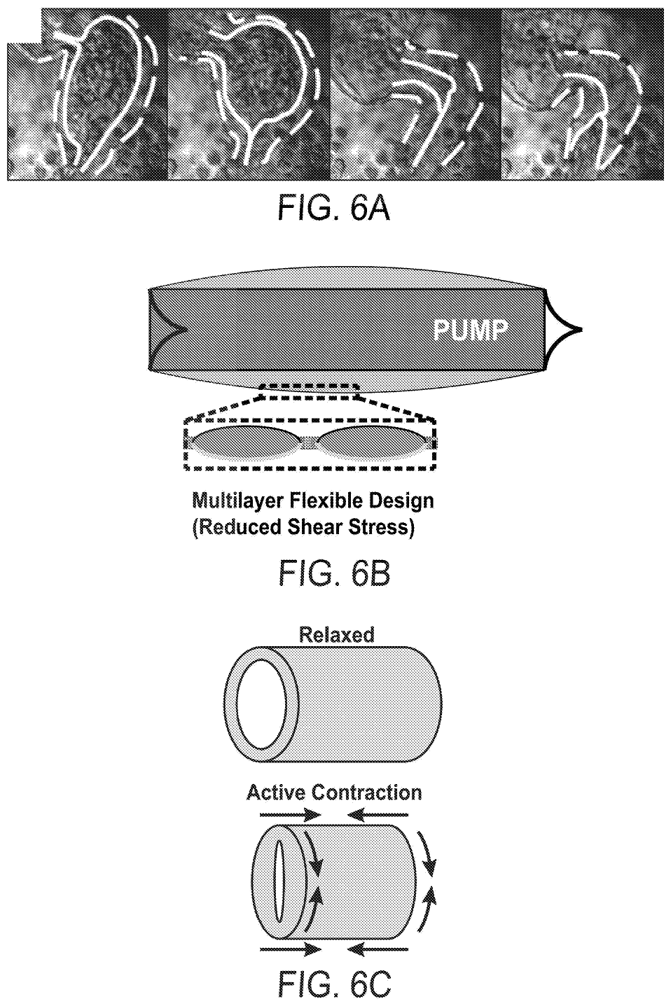

The pump 100 can include a controller operably coupled to the electrode sets 112. The controller can include a processor and memory operably coupled to the processor (e.g., at least the processing unit and system memory of computing device 500 of FIG. 5). Additionally, the pump 100 can include a power supply (e.g., a battery) operably coupled to the electrode sets 112. The controller can be configured to stimulate one or more of the electrode sets 112 in a predetermined pattern. Alternatively or additionally, the controller can be configured to sense mechanical strain using one or more of the electrode sets 112. As described herein, when a voltage is applied to an electrode set 112, the IPMC 110 (or other electrically active material) is activated, which causes a pouch 108 to deform. It should be understood that stimulating the one or more of the electrode sets 112 can cause one or more of the pouches 108 to deform. Deforming the one or more pouches 108 can cause circumferential and/or longitudinal contraction of the outer tubular structure 104, which can pump the biological fluid 200 through the inner tubular structure 102. In some implementations, the one or more of the electrode sets 112 can be stimulated in the predetermined pattern to cause wavelike action in the outer tubular structure 104. In other implementations, the one or more of the electrode sets 112 can be stimulated in the predetermined pattern to cause peristaltic action in the outer tubular structure 104. In yet other implementations, the one or more of the electrode sets 112 can be stimulated in the predetermined pattern to cause non-peristaltic action in the outer tubular structure 104. The wavelike, peristaltic, or non-peristaltic action can move the biological fluid 200 from one end of the pump 100 to the other (e.g., from the proximal to the distal end of the pump 100). In yet further implementations, the one or more of the electrode sets 112 can be stimulated in the predetermined pattern to simultaneously contract the entire outer tubular structure 104. Simultaneous contraction of the entire outer tubular structure 104 can move a bolus of the biological fluid 200 through the pump 100.

In some implementations, the pump 100 can include a pneumatic or hydraulic pump arranged in fluid connection with the pouches 108. In other words, the pouches 108 can be driven (e.g., expanded/relaxed) using pressurized air or fluid. This is in contrast to the implementation where each pouch 108 forms an electrohydraulic transducer. This disclosure contemplates that the controller, e.g., a processor and memory operably coupled to the processor (e.g., at least the processing unit and system memory of computing device 500 of FIG. 5), can be operably coupled to the pneumatic or hydraulic pump. The controller can actuate the pneumatic or hydraulic pump, where actuating the pneumatic or hydraulic pump causes one or more of the pouches 108 to deform. This disclosure contemplates that the pneumatic or hydraulic pump can be actuated to deform the pouches 108 to achieve the fluid flow as described herein.

The pump 100 can be configured such that a maximum internal shear stress experienced by the biological fluid 200 is about 60 dynes/cm² or less (e.g., <60 dynes/cm², <55 dynes/cm², <50 dynes/cm², . . . <5 dynes/cm²) and any value or range therebetween; with an average of less than 10 dynes/cm². Optionally, the pump 100 can be configured such that the maximum internal shear stress experienced by the biological fluid 200 is about 30 dynes/cm² or less (e.g., <30 dynes/cm², <25 dynes/cm², <20 dynes/cm², . . . <5 dynes/cm²) and any value or range therebetween; with an average of less than 10 dynes/cm². As used herein, the maximum internal shear stress is measured in the compressing expanding region of the pump 100. The compressing expanding region is the region within the pump 100 (i.e., where the biological fluid 200 flows) within the inner tube or lumen, excluding shear stress values found on any potential valves. Additionally, the prosthetic valve provided on the distal (downstream) end (e.g., one of prosthetic valves 114 in FIG. 1) of the pump 100 can reach a maximum shear stress of 80 dynes/cm² or less (e.g., <80 dynes/cm², <75 dynes/cm², <70 dynes/cm², . . . <5 dynes/cm²) and any value or range therebetween. In contrast, commercial continuous flow ventricular assist devices (VADs), e.g. axial flow and centrifugal pumps, exert high internal shear stress (e.g., shear stress >500 dynes/cm²) on the blood flowing through. Pulsatile VADs, which are rarely used, exert less shear stress on blood as compared to continuous flow VADs, but this shear stress can still exceed 40 dynes/cm² in the primary body of the device, and pulsatile VADs typically involve a mechanical heart valve, which can reach a shear stress of 1500 dynes/cm² and can lead to thrombus formation. For comparison, the approximate maximum shear stress experienced by blood flowing through a human circulatory system is about 150 dynes/cm² in arterioles, while the approximate maximum shear stress experienced at the heart is 80 dynes/cm² at the aortic valve.

In some implementations, the pump 100 can be configured such that a maximum internal shear stress experienced by the biological fluid 200 is less than the maximum internal shear stress experienced by blood flowing through a continuous flow VAD, which can exceed 500 dynes/cm². It should be understood that 500 dynes/cm² is provided only as an example and that this disclosure contemplates that the biological fluid can experience a maximum internal shear stress of less than 500 dynes/cm², 400 dynes/cm², 300 dynes/cm², 200 dynes/cm², or 100 dynes/cm², etc. In other implementations, the pump 100 can be configured such that a maximum internal shear stress experienced by the biological fluid 200 is less than the maximum shear stress experienced by blood flowing through the circulatory system of a human, which is about 150 dynes/cm² in the arterioles and about 80 dynes/cm² in the heart. Because maximum internal shear stress using the pump 100 described herein can be about (or even less than) the upper physiological values, the risk of non-surgical bleeding, thrombosis, and/or stroke can be reduced as compared to when using a conventional VAD. As noted above, continuous flow VADs exert very high shear stress on blood, while pulsatile VADs exert lower shear stress on blood but typically include a mechanical heart valve where maximum shear stress can reach 1500 dynes/cm². On the other hand, the maximum internal shear stress exerted on the biological fluid by the pump 100 described herein is about 60 dynes/cm² or less, while maximum shear stress at the distal prosthetic value can be about 80 dynes/cm² or less, i.e., both maximum shear stress values are less than the upper physiological value for shear stress experienced by blood in a human body. It should be understood that 150 dynes/cm² is provided only as an example and that this disclosure contemplates that the biological fluid can experience a maximum internal shear stress of less than 140 dynes/cm², 130 dynes/cm, 120 dynes/cm², 110 dynes/cm², 100 dynes/cm², 90 dynes/cm², 80 dynes/cm², 70 dynes/cm², 60 dynes/cm², or 50 dynes/cm², etc. In yet other implementations, the pump 100 can be configured such that a maximum internal shear stress experienced by the biological fluid 200 is less than the maximum internal shear stress experienced by blood flowing through a pulsatile pump, which is about 40 dynes/cm². In yet other implementations the biological fluid 200 can experience a maximum internal shear stress of less than 30 dynes/cm², such as 25 dynes/cm², 20 dynes/cm², 15 dynes/cm², 10 dynes/cm², or 5 dynes/cm² while flowing through the pump 100.

It should be understood that continuous flow pumps are more commonly used in conventional VADs. Pulsatile pumps are rarely used, compared to continuous flow pumps since pulsatile pumps suffer from durability complications from multiple moving parts, with specific issues associated with bearings. Pulsatile pumps are also heavy. In contrast, the pump 100 described herein can have one or more moving fluid chambers (e.g., depending on the arrangement of the pouches 108 in FIG. 1) and no bearings. Additionally, the fluid chamber durability of pump 100 described herein is similar to the durability of polymeric prosthetic heart valves, which have been proven to be highly durable. The design of pump 100 also dramatically reduces the weight below that of pulsatile or continuous flow pumps since metal is more dense than the materials (e.g., polymers) used in the pump 100 described herein.

Alternatively or additionally, the pump 100 can include at least one prosthetic valve 114 arranged in fluid connection with the inner tubular structure 102. As shown in FIG. 1, the pump 100 can include two prosthetic valves 114, e.g., a prosthetic valve 114 at each end of the pump 100. Alternatively or additionally, at least a portion of the inner tubular structure 102 can extend outside of the outer tubular structure 104. Additionally, the prosthetic valve 114 can be arranged in the portion of the inner tubular structure 102 that extends outside of the outer tubular structure 104. Optionally, the prosthetic valve 114 can be configured to prevent backflow of the biological fluid 200. In some implementations, the prosthetic valve 114 can include at least one leaflet. For example, a tilting disk mechanical heart valve has a single leaflet (e.g., a single disk restrained by struts). In other implementations, the prosthetic valve 114 can include a plurality of leaflets. For example, bicuspid and trileaflet prosthetic valves have a plurality of leaflets. Optionally, the leaflet(s) can define a sinus region.

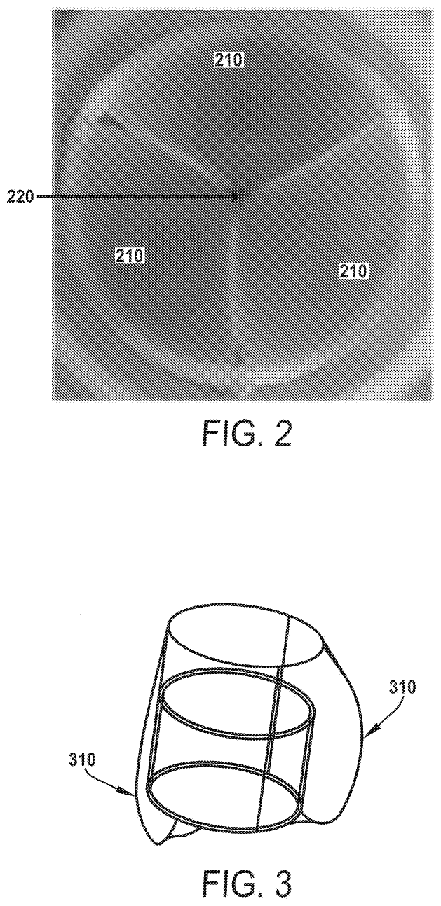

In some implementations, the prosthetic valve 114 can be a synthetic prosthetic valve that is intended to mimic geometry of the native human aortic valve, similar to fabric-based valves and polymeric valves that are known in the art. The human aortic valve is a trileaflet valve, meaning that it has three (3) leaflets. Thus, the prosthetic valve 114 can be designed in the same way. FIG. 2 illustrates an example trileaflet prosthetic valve. The leaflets 210 (e.g., three leaflets) come together at a commissure 220, preventing back flow when pressure at the leaflet tip exceeds the pressure upstream/proximal of the valve.

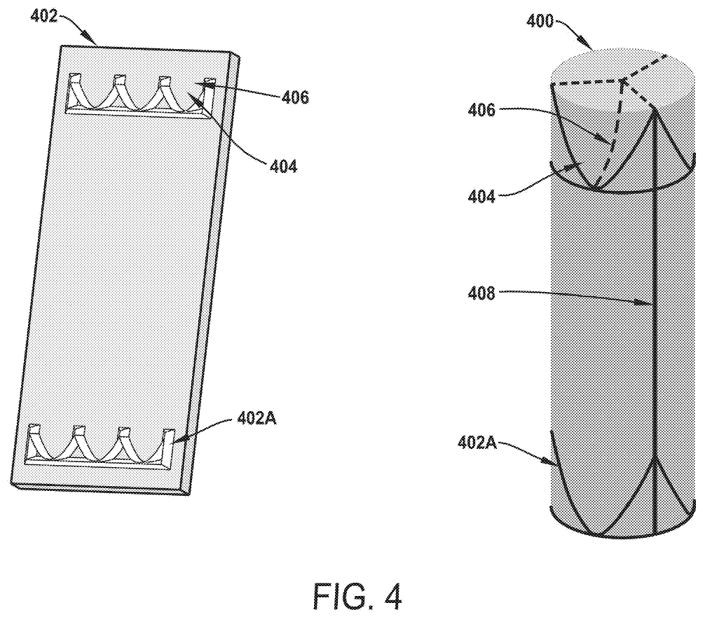

When pressure is reversed, the trileaflet prosthetic valve opens into a cylinder-like shape, which is shown by FIG. 3. One risk for valves is coagulation, which can occur in regions where blood stagnates. To avoid stagnation, a ‘sinus’ region can be created around each leaflet, similar to the aortic sinus distal/downstream of the native aortic valve. The sinus is shown as the outer curvature region 310 in FIG. 3. With the sinus, a fluid vortex can form between the leaflets and the sinus, creating a ‘washout’ of blood between the leaflets and outer wall. In the case of the pump, the outer wall or sinus is the inner lumen (e.g., the inner tubular structure 102 in FIG. 1) of the pump, which exists inside the gelatin layer (e.g., the gel layer 106 in FIG. 1) and artificial muscle layer (e.g., the outer tubular structure 104 in FIG. 1) of the pump. Thus, shear stress, which can cause hemolysis or can activate platelets, can be minimized by creating this geometry that mimics the native valve. This design maximizes the effective orifice area of the valve when it's open, minimizing shear stress.

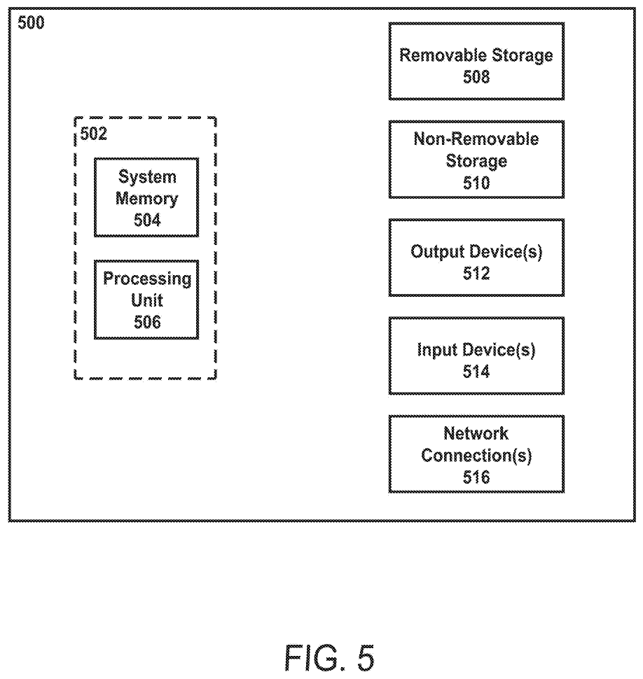

In some implementations, the valve geometry can be created on flat sheets of polymer 400 by creating a bond between polymer sheets (valve leaflets and pump lumen). As described above, a thermal weld can be created between two sheets of polymer, using a heated metal block 402, or similar approach, with an example geometry of the ‘stencil’ shown in FIG. 4. A ridge/rise/projection 402A can be used to create a welded seam in the polymer. After joining of the polymer sheets, the polymer sheets can be rolled into a cylinder-like shape, thereby creating the valve geometry described above. A second (final) seam 408 can be created to finalize the cylindrical geometry. The sinus region 404 of the valve can be formed using a trough-like region between welding points, as shown in FIG. 4, along the outer cylinder or polymer layer opposite of the leaflet cusp 406 existing on the inner cylinder or polymer layer. This allows for excess material, which can be used to form the sinus. As shown in FIG. 4, valves, including the sinus geometry can be created at both ends of the pump design to prevent regurgitant/reverse/backward flow in the pump. The artificial muscle can then be created between the valves. The artificial muscle can be used to constrict the inner lumen of the pump.

It should be appreciated that the logical operations described herein with respect to the various figures may be implemented (1) as a sequence of computer implemented acts or program modules (i.e., software) running on a computing device (e.g., the computing device described in FIG. 5), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 5, an example computing device 500 upon which embodiments of the invention may be implemented is illustrated. It should be understood that the example computing device 500 is only one example of a suitable computing environment upon which embodiments of the invention may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in FIG. 5 by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

An example ventricular assist device pump is described herein. The VAD includes a flexible inner tube or lumen (e.g., the inner tubular structure 102 in FIG. 1) including prosthetic heart valves, a gel layer (e.g., the gel layer 106 in FIG. 1), and an artificial muscle (e.g., the cuter tubular structure 104 in FIG. 1). Ventricular assist devices help to drive blood flow and are used, for example, during heart failure. Conventional VADs suffer from bleeding and thrombosis (blood clots), which are in-part due to higher shear stress (harsh flow conditions). The flexible VADs described herein can reduce, minimize, and/or eliminate these issues.

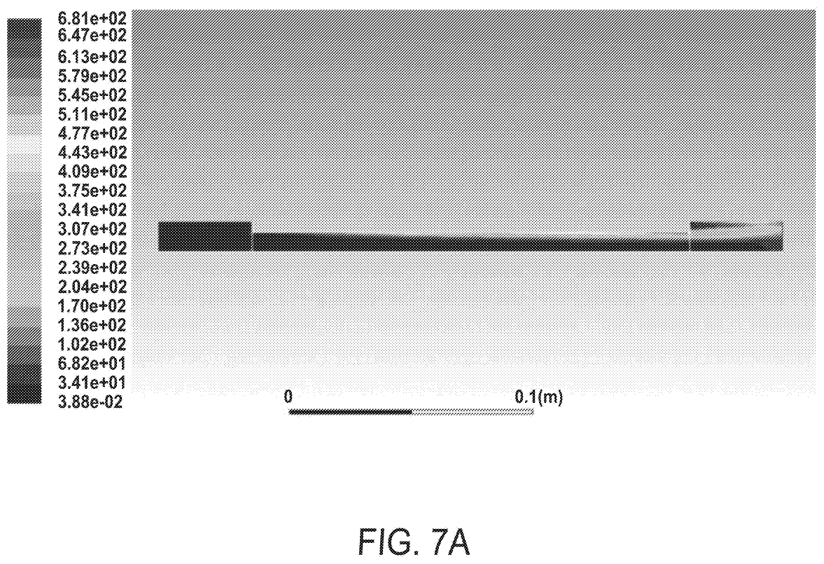

A consistent finding in VADs is that high shear stress associated with VAD dynamics can lead to bleeding. Furthermore, continuous flow applied at the aorta from VADs can lead to vascular remodeling. The flexible pump described below, which offers shear stress comparable to physiological conditions, can greatly reduce the risk of non-surgical bleeding. Inspired by the early embryonic heart (prior to 4 chambers or valves), which is shown in FIG. 6A, a flexible pump design has been developed that leverages hydraulic forces. The flexible pump is shown in FIG. 6B. Similar hydraulic forces are receiving substantial use in the field of soft robotics due to their delicate handling of objects, potential for self-healing, reduced power consumption, and silent operation. The flexible pump design leverages these concepts to remove the rotating mechanical parts associated with commercially available VADs. Also, shear stress is reduced closer to (or even below) physiological values, since flow rates would not exceed normal cardiac output and since the narrowest section of the flexible pump incorporates a bioprosthetic valve that is the same size as native heart valves.

The flexible pump design includes a gel layer (e.g., the gel layer 106 in FIG. 1) sandwiched between an inner tube (e.g., the inner tubular structure 102 in FIG. 1) and artificial muscle (e.g., the outer tubular structure 104 in FIG. 1) controlled by a series of electrohydraulic transducers that linearly contract, similar to a pneumatic soft robotic design. Resultantly, there is a large change in compression of the inner lumen of the tube for small changes in length of the artificial muscle. This is shown in FIG. 6C. This design is sometimes referred to herein as the Pulsatile Undulating Multilayered Pump (PUMP). The PUMP design can create a peristaltic (undulating)-type motion in the flow direction, with hemodynamics similar to the native ventricle. This design can improve the quality of life for many patients who use VADs or other blood-contacting pumps, e.g. extracorporeal membrane oxygenation circuits.

The PUMP design mimics dynamics of the early heart to create an electrohydraulic driven Pulsatile Undulating Multilayered Pump (PUMP), which includes a gelatin (Sigma Aldrich) layer sandwiched between a flexible tube and an outer artificial muscle. These pumps are very inexpensive to fabricate. The artificial muscle can include a flexible inextensible shell made from transparent biaxially-oriented polypropylene (BOPP), as utilized for soft robotics, or other commercially available polymer. Hydraulic pouches can be created by thermally welding a plurality (e.g., 2) layers of BOPP in a cylindrical shape, using a combination of an aluminum bar and a heated brass die to create circumferentially and axially spaced hollow pouches along the tube. Axial spacing allows a peristaltic-like motion, moving blood from the proximal to the distal end of the pump. Channels can be left in the pouches to fill with a fluid such as saline, which can be subsequently heat sealed. Ionic polymer-metal composites (IPMCs) (Environmental Robots Inc.) can be affixed over the pouches on the BOPP sleeve. The electrodes of the IPMC can be connected to a power supply such as a battery. IPMCs create a bending motion within microseconds (μs) when a low driving voltage (<5 V) is applied across the thickness, causing pouches to ‘round’ after pouch attachment. The electrodes of the IPMC can be connected to a power supply controller to dictate pumping. IPMCs can also be used for sensing mechanical strain, which will be utilized for a control system that will drive the PUMP. Overall, the combined IPMC and BOPP layers create the artificial muscle layer of the PUMP.

Separately, for the inner tube, a strip of polymer (e.g., BOPP) can be welded onto another polymer sheet, with the shape of 3 heart valve leaflets (e.g., each of the leaflets with a U-shaped geometry). This can be repeated for the other side of the polymer sheet, for example, to form prosthetic valves at opposite ends of the inner tube. The polymer sheet can be used as the inner lumen of the pump, while the leaflet-shaped geometry serves as a valve. This polymer sheet can be shaped into a cylinder, while the shorter outer artificial muscle can be wrapped around the inner tube, leaving the valve/leaflets outside of (i.e., extending beyond the end of) the artificial muscle area.

The inner tube can be heat welded at the ends to the outer artificial muscle. The gap can be filled with a gel layer such as gelatin. These layers enhance and homogenize the volume displacement of the pump. As the artificial muscle deforms by ‘rounding’, it causes circumferential and longitudinal contraction, similar to the native myocardial layer. The reduction in diameter and volume displacement in the pouches displaces the incompressible gel layer, which compresses the inner tube, as shown in FIG. 6C. The pump can be combined with 2.5 cm bioprosthetic heart valves affixed inside either end of the inner tube to ensure unidirectional flow.

The presence of the gel layer results in a much larger reduction of the inner diameter of the inner tube, compared to a straight single layer tube, a result seen in the embryonic heart. In the example design, the inner volume changes by a factor of t/R(1/λ²−1)(2+t/R) more than if the gel layer was absent for a given λ, where t is the thickness of the gelatin, and R is radius of the inner tube. λ≅2/π defines the approximate maximum circumference change based on pouch length change (assuming flattening in diastole and full rounding during systole). With a pump length of 15 cm, a 70 milliliter (ml) stroke volume and 145 ml end diastolic volume can be achieved using an inner tube radius of 1.7 cm and an outer tube radius of 2 cm. Additionally, matching conditions of the heart, 0 to 10 liters/min flow can be achieved, while providing 40-120 mmHg pressure.

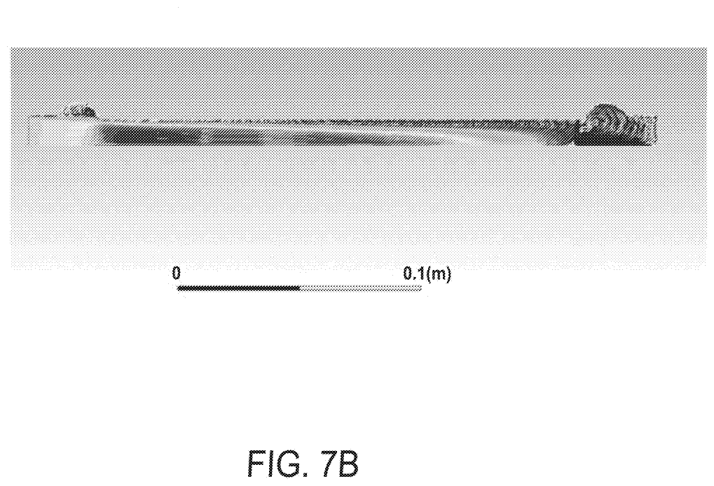

Referring now to FIGS. 7A and 7B, graphs illustrating simulated shear stress (FIG. 7A) and flow (FIG. 7B) within an example flexible pump according to implementations described herein are shown. With reference to FIG. 7A, a maximum internal shear stress in the flexible pump (e.g., flexible pump 100 shown in FIG. 1) is estimated to be about 28 dynes/cm² during systole under conditions that match physiological conditions during systole. The simulation involved idealized conditions, where the entire inner wall (e.g., inner tubular structure 102 shown in FIG. 1) moves radially to produce a physiological cardiac output. A wall boundary condition was applied to the left side, simulating a closed valve, while a pressure outlet condition was applied on the right, simulating an open valve. A contour plot of strain rate is shown in FIG. 7A. The results are axisymmetric, meaning that the bottom boundary is a central axis, while the top boundary is rotated circumferentially. The flow in the cylinder is equal regardless of circumferential location. FIG. 7A shows planar representation from the center of the cylinder to the outer wall. The simulation was repeated with a sinus (e.g., bulbous region in the figure). These results are shown in FIG. 7B. This allows flow to washout regions behind valve leaflets. In FIG. 7B, velocity vectors are shown with contours corresponding to the velocity magnitude. The same idealized moving wall is assumed in this simulation. The maximum internal shear stress or strain rate remained unchanged with or without a sinus region.

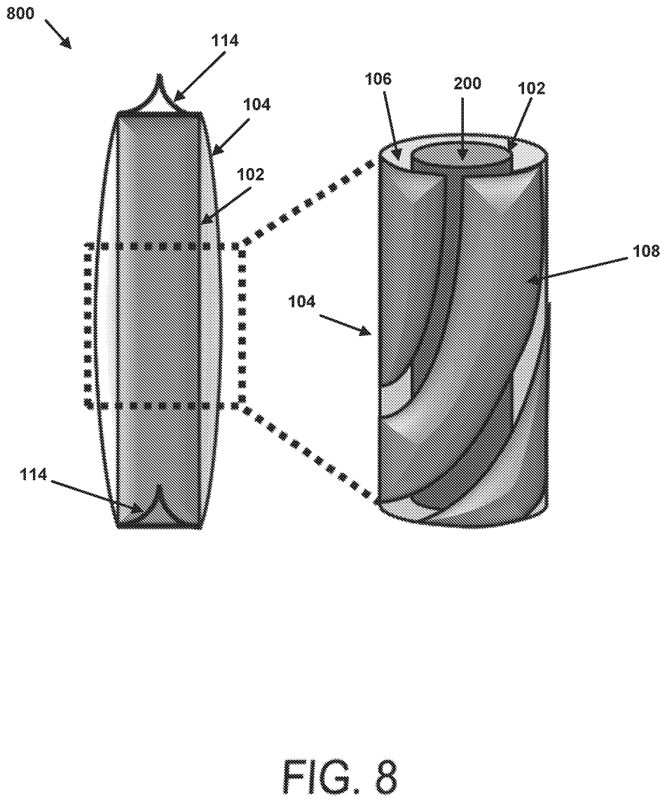

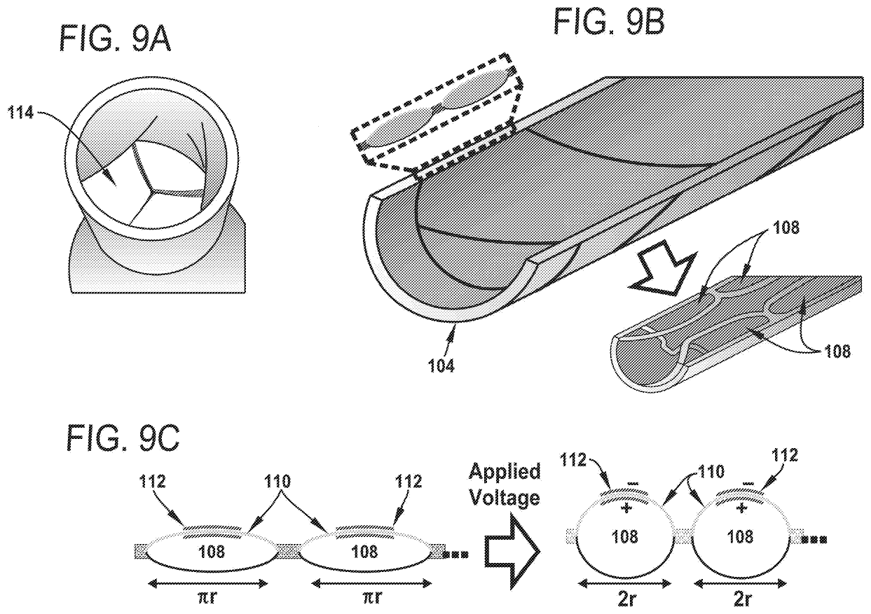

In some implementations, the flexible pump can include a plurality of pouches arranged in a spiral pattern. This is in contrast to a pump having pouches arranged in a grid-like pattern as shown in FIG. 1. Referring now to FIGS. 8-10B, a flexible pump having a plurality of pouches arranged in a spiral pattern is described. As shown in FIGS. 8-9C, the flexible pump 800 can include the inner tubular structure 102, the outer tubular structure 104, the gel layer 106, and the prosthetic valve 114. The flexible pump 800 can be configured to drive the biological fluid 200 as described herein. For example, in some implementations, each of the pouches 108 can be an electrohydraulic transducer (e.g., having electrode sets 112 and IPMC layer 110). In other implementations, each of the pouches 108 can be driven (e.g., expanded/relaxed) using pressurized air or fluid. Expansion/relaxation of the pouches 108 is shown, for example, in FIG. 9B. Additionally, as shown in FIG. 8, the pouches 108 are arranged in a spiral pattern. The spiral pattern can be designed with various pitches. In some implementations, the pitch of the spiral is the same throughout the device, while in other implementations the pitch of the spiral is different in different sections of the device. This disclosure contemplates that the size/shape of the pouches 108, arrangement, and/or spacing between the pouches 108 can be different than the example shown in FIG. 8.

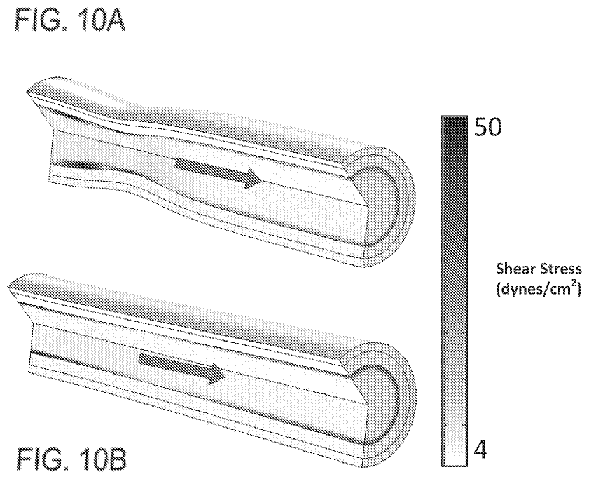

FIGS. 10A and 10B are FSI models of fluid shear stress for fluid flowing through the inner tubular structure of a flexible pump. In FIG. 10A, the pouches are circumferentially aligned such that continuous pouches extend around the complete circumference of the tube while being spaced along the axis of the tube. In FIG. 10B, the pouches are axially aligned such that continuous pouches extend from one end of the tube to the other, while being spaced circumferentially. The axially aligned design (e.g., FIG. 10B) reduces the necessary internal shear stress relative to a circumferentially arranged pouch configuration (e.g. FIG. 10A) for the pump to achieve a specific stroke volume during a single cycle of actuation.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A pump for driving a biological fluid, comprising:
an inner tubular structure;
an outer tubular structure arranged around the inner tubular structure, wherein the outer tubular structure is configured as an artificial muscle; and
a gel layer disposed between the inner and outer tubular structures.

2. The pump of claim 1, wherein at least one of the inner tubular structure, the outer tubular structure, or the gel layer is flexible.

3. The pump of claim 1, wherein each of the inner tubular structure, the outer tubular structure, and the gel layer are flexible.

4. The pump of claim 1, wherein each of the inner tubular structure, the outer tubular structure, and the gel layer comprise biocompatible material.

5. The pump of claim 1, wherein the inner tubular structure comprises an extensible flexible polymer or an inextensible flexible polymer.

6. The pump of claim 1, wherein the outer tubular structure comprises an inextensible flexible polymer.

7. The pump of claim 1, wherein the pump is configured such that a maximum internal shear stress experienced by the biological fluid is about 150 dynes/cm² or less.

8. (canceled)

9. The pump of claim 1, wherein the gel layer comprises a liquid, a liquid hydrogel, an organogel, a gelatin, an animal produced gel, or combinations thereof.

10. The pump of claim 1, wherein the gel layer has a thickness between about 0.1-2 cm.

11. (canceled)

12. The pump of claim 1, wherein the outer tubular structure comprises a plurality of pouches, wherein each of the pouches is filled with fluid.

13. The pump of claim 12, wherein the pouches are spaced apart from one another in an axial direction and/or a circumferential direction along the outer tubular structure.

14. (canceled)

15. The pump of claim 12, wherein the outer tubular structure comprises a plurality of polymer layers.

16. (canceled)

17. (canceled)

18. The pump of claim 12, wherein each of the pouches is configured to deform in response to an external stimulus.

19. (canceled)

20. The pump of claim 12, further comprising a plurality of electrode sets, wherein each respective electrode set is disposed on a respective one of the pouches.

21. The pump of claim 20, wherein each respective electrode set and pouch forms an electrohydraulic transducer.

22. (canceled)

23. The pump of claim 20, further comprising a controller operably coupled to the electrode sets, wherein the controller comprises a processor and memory operably coupled to the processor, the memory having computer-executable instructions stored thereon that, when executed by the processor, cause the controller to stimulate one or more of the electrode sets in a predetermined pattern.

24. The pump of claim 23, wherein stimulating the one or more of the electrode sets in the predetermined pattern is configured to cause motion of the outer tubular structure.

25. (canceled)

26. (canceled)

27. (canceled)

28. The pump of claim 20, further comprising a controller operably coupled to the electrode sets, wherein the controller comprises a processor and memory operably coupled to the processor, the memory having computer-executable instructions stored thereon that, when executed by the processor, cause the controller to stimulate one or more of the electrode sets, wherein stimulating the one or more of the electrode sets causes one or more of the pouches to deform.

29. The pump of claim 28, wherein deforming the one or more of the pouches causes circumferential and/or longitudinal contraction of the outer tubular structure.

30. (canceled)

31. The pump of claim 23, wherein the memory has further computer-executable instructions stored thereon that, when executed by the processor, cause the controller to sense mechanical strain using one or more of the electrode sets.

32. (canceled)

33. The pump of claim 12, further comprising a pneumatic or hydraulic pump arranged in fluid connection with the pouches.

34. (canceled)

35. The pump of claim 1, further comprising at least one prosthetic valve arranged in fluid connection with the inner tubular structure.

36. The pump of claim 35, wherein the at least one prosthetic valve is configured to prevent backflow of the biological fluid.

37. The pump of claim 35, wherein the at least one prosthetic valve comprises at least one leaflet.

38. (canceled)

39. (canceled)

40. The pump of claim 35, wherein at least a portion of the inner tubular structure extends outside of the outer tubular structure, and wherein the at least one prosthetic valve is arranged in the portion of the inner tubular structure that extends outside of the outer tubular structure.

41. (canceled)

42. (canceled)

43. (canceled)
